(19) United States
(12) Patent Application Publication
(10) Pub. No.: US 2025/0073030 A1
(43) Pub. Date: Mar. 6, 2025

(54) OUTER SKIRT FOR AN EXPANDABLE PROSTHETIC HEART VALVE

(71) Applicant: EDWARDS LIFESCIENCES CORPORATION, Irvine, CA (US)

(72) Inventor: David Maimon, Atlit (IL)

(21) Appl. No.: 18/952,970

(22) Filed: Nov. 19, 2024

Related U.S. Application Data
(63) Continuation of application No. PCT/US2023/024761, filed on Jun. 7, 2023.
(60) Provisional application No. 63/351,238, filed on Jun. 10, 2022.

(51) Int. Cl. A61F 2/24

(52) U.S. Cl. CPC A61F 2/2418; A61F 2/2433

(57) ABSTRACT

Extension members for an outer skirt of a prosthetic heart valve and methods for unfolding the extension members during radial expansion of the prosthetic heart valve are disclosed. As one example, a prosthetic heart valve comprises a frame that is radially expandable and compressible between a radially compressed configuration and a radially expanded configuration, and an outer skirt disposed around an outer surface of the frame. The outer skirt comprises one or more extension members that are movable between a first state where the one or more extension members are folded against the frame and a second state where the one or more extension members extend radially outward and away from the frame, and where the one or more extension members are in the first state when the frame is in the radially compressed configuration and in the second state when the frame is in the radially expanded configuration.

### CROSS-REFERENCE TO RELATED APPLICATIONS

This application is a continuation of PCT Application No. PCT/US2023/024761, filed Jun. 7, 2023, which claims the benefit of U.S. Provisional Patent Application No. 63/351,238, filed Jun. 10, 2022, the entire contents of each of which are incorporated by reference herein.

### FIELD

The present disclosure relates to prosthetic heart valves, and in particular to outer coverings or skirts for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable, prosthetic heart valves comprise a cylindrical metal frame or stent and prosthetic leaflets mounted inside the frame. These valves can also include one or more coverings (or skirts) spanning a circumference of the frame, on an inner or outer surface of the frame. These coverings can be configured to establish a seal with the native tissue when the prosthetic heart valve is placed at the implantation site (and thus may be referred to as sealing members). However, the native tissue (for example, at the native valve annulus or arterial wall around the native valve) can have an irregular shape while the frame of the prosthetic heart valve is generally cylindrical. As a result, gaps can be formed between the prosthetic heart valve and native heart valve annulus when the prosthetic heart valve is implanted within the native heart valve annulus, even when coverings are included on the prosthetic heart valve.

Accordingly, a need exists for improved coverings or outer skirts for prosthetic heart valves which can better fills gaps between the native tissue and the prosthetic heart valve.

### SUMMARY

Described herein are prosthetic heart valves, delivery apparatuses, and methods for implanting prosthetic heart valves. In particular, described herein are examples of coverings for a prosthetic heart valve and methods of making and using such coverings. Prosthetic heart valves can include a frame and a leaflet assembly arranged on an inner surface of the frame. The prosthetic heart valve can include a covering (or outer skirt) arranged around a circumference of the frame and on an outer surface of the frame. The outer skirt can include one or more flexible extension members that protrude radially outward and away from the frame when the prosthetic heart valve is in a radially expanded configuration. The one or more flexible extension members can be configured such that the prosthetic heart valve, once implanted, better conforms to a shape of the surrounding native tissue. Further, in some examples, the one or more flexible extension members can be configured to be disposed in a folded configuration, against the frame, when the prosthetic heart valve is in a radially compressed configuration. Then, as the prosthetic heart valve radially expands (for example, during deployment at the implantation site), the one or more flexible extension members can unfold into their radially protruding configuration. As such, the skirts and prosthetic heart valves disclosed herein can, among other things, overcome one or more of the deficiencies of typical prosthetic heart valves.

A prosthetic heart valve can comprise a frame and a valvular structure coupled to the frame. In addition to these components, a prosthetic heart valve can further comprise one or more of the components disclosed herein.

In some examples, the prosthetic heart valve can comprise a sealing member configured to reduce paravalvular leakage.

In some examples, the sealing member is an outer skirt disposed around an outer surface of the frame.

In some examples, the outer skirt comprises one or more extension members that are movable between a first state where the one or more extension members are folded against the frame and a second state where the one or more extension members extend radially outward and away from the frame.

In some examples, the one or more extension members are in the first state when the frame is in the radially compressed configuration and in the second state when the frame is in the radially expanded configuration.

In some examples, the one or more extension members includes a plurality of extension members spaced circumferentially apart from each other around the outer skirt.

In some examples, the outer skirt comprises an inflow edge secured to an inflow end of the frame and an outflow edge secured to an intermediate portion of the frame that is disposed between the inflow end and an outflow end of the frame.

In some examples, each extension member of the one or more extension members extends between the inflow edge and the outflow edge of the outer skirt.

In some examples, a prosthetic heart valve comprises an annular frame that is radially expandable and compressible between a radially compressed configuration and a radially expanded configuration, and an outer skirt disposed around an outer surface of the frame. The outer skirt comprises one or more extension members that are movable between a first state where the one or more extension members are folded against the frame and a second state where the one or more extension members extend radially outward and away from the frame. The one or more extension members are in the first state when the frame is in the radially compressed configuration and in the second state when the frame is in the radially expanded configuration.

In some examples, a prosthetic heart valve comprises a radially expandable and compressible annular frame configured to move between a radially compressed state and a radially expanded state, and an outer skirt disposed around an outer surface of the frame. The outer skirt comprises a first portion disposed against the outer surface of the frame and a second portion comprising a plurality of extension members spaced circumferentially apart from each other around the outer skirt. The plurality of extension members is configured to be folded against the first portion when the frame is in the radially compressed state and unfold to extend radially outward from the frame when the frame is radially expanded from the radially compressed state to the radially expanded state.

In some examples, a prosthetic heart valve comprises one or more of the components recited in Examples 1-26, and 61 below.

An assembly can comprise a delivery apparatus comprising an inflatable balloon, and a prosthetic heart valve mounted around the balloon in a radially compressed configuration.

In some examples, the prosthetic heart valve can comprise an annular frame and an outer skirt disposed around an outer surface of the frame.

In some examples, the outer skirt can comprise a plurality of extension members, where the plurality of extension members is folded against the frame when the frame is in the radially compressed configuration and are configured to unfold to extend radially outward from the frame when the frame is radially expanded from the radially compressed configuration to a radially expanded configuration by inflation of the balloon.

In some examples, the extension members of the plurality of extension members are spaced circumferentially apart from each other around the outer skirt.

In some examples, an assembly comprises an inflatable balloon disposed at a distal end portion of a delivery apparatus, and a prosthetic heart valve mounted around the balloon in a radially compressed configuration. The prosthetic heart valve comprises an annular frame and an outer skirt disposed around an outer surface of the frame. The outer skirt comprises a plurality of extension members spaced circumferentially apart from each other around the outer skirt, where the plurality of extension members are folded against the frame when the frame is in the radially compressed configuration and are configured to unfold to extend radially outward from the frame when the frame is radially expanded from the radially compressed configuration to a radially expanded configuration by inflation of the balloon.

In some examples, an assembly comprises one or more of the components recited in Examples 39-48 below.

A method can comprise advancing a prosthetic heart valve that is radially compressed around an inflatable balloon of a distal end portion of a delivery apparatus to an implantation site using the delivery apparatus.

In some examples, the prosthetic heart valve comprises an annular frame and an outer skirt disposed around an outer surface of the frame, and where the outer skirt comprises extension members that are folded against the frame.

In some examples, the method can comprise inflating the balloon of the delivery apparatus at the implantation site to radially expand and implant the prosthetic heart valve, and unfolding the extension members to protrude radially outward and away from the frame as the prosthetic heart valve radially expands.

In some examples, the method can comprise radially expanding and rotating the prosthetic heart valve in a first direction as it radially expands and unfolding the extension members in a second direction that is opposite the first direction in response to the rotating such that the extension members protrude radially outward from the frame when the prosthetic heart valve in a radially expanded configuration.

In some examples, a method comprises advancing a prosthetic heart valve that is radially compressed around an inflatable balloon of a distal end portion of a delivery apparatus to an implantation site using the delivery apparatus, where the prosthetic heart valve comprises an annular frame and an outer skirt disposed around an outer surface of the frame, and where the outer skirt comprises extension members that are folded against the frame. The method further comprises inflating the balloon of the delivery apparatus at the implantation site to radially expand and implant the prosthetic heart valve, and unfolding the extension members to protrude radially outward and away from the frame as the prosthetic heart valve radially expands.

In some examples, a method comprises radially expanding and rotating a prosthetic heart valve in a first direction as it radially expands by inflating a balloon of a delivery apparatus, where the prosthetic heart valve is mounted around the balloon in a radially compressed configuration prior to inflating the balloon, and where the prosthetic heart valve comprises an annular frame and an outer skirt disposed around an outer surface of the frame, the outer skirt comprising extension members that are folded against the frame. The method further comprises unfolding the extension members in a second direction that is opposite the first direction in response to the rotating such that the extension members protrude radially outward from the frame when the prosthetic heart valve in a radially expanded configuration.

The above method(s) can be performed on a living animal or on a simulation, such as on a cadaver, cadaver heart, anthropomorphic ghost, simulator (e.g., with body parts, heart, tissue, etc. being simulated).

In some examples, a method comprises one or more of the components recited in Examples 37-38, 49-60, and 62 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of an example of a delivery apparatus configured to deliver and implant a radially expandable prosthetic heart valve at an implantation site.

FIG. 3 is a cross-sectional view of an inflatable balloon wrapped and folded around a portion of a delivery apparatus, at a valve mounting portion of the delivery apparatus, according to an example.

FIGS. 4A-4C are schematic cross-sectional views illustrating rotation of a frame of a prosthetic heart valve during radial expansion of the prosthetic heart valve with an inflatable balloon.

FIGS. 5A-5C are schematic side views illustrating the rotation of the frame of the prosthetic heart valve of FIGS. 4A-4C during inflation of the balloon and radial expansion of the prosthetic heart valve.

FIGS. 6A-6C are schematic cross-sectional views illustrating rotation of a frame of a prosthetic heart valve in a first direction as it is radially expanded by inflation of a balloon of a delivery apparatus and unfolding of extension members of an outer skirt of the prosthetic heart valve in a second direction as the frame radially expands and rotates.

FIG. 7 is a perspective view of a prosthetic heart valve including an outer skirt disposed around an outer surface of a frame of the prosthetic heart valve, the outer skirt comprising foldable extension members, according to one example, that protrude radially outward from the frame when the prosthetic heart valve is in a radially expanded state.

FIG. 8 is a perspective view of a prosthetic heart valve including an outer skirt disposed around an outer surface of a frame of the prosthetic heart valve, the outer skirt comprising foldable extension members, according to an example, that protrude radially outward from the frame when the prosthetic heart valve is in a radially expanded state.

FIG. 9 is a perspective view of a prosthetic heart valve including an outer skirt disposed around an outer surface of a frame of the prosthetic heart valve, the outer skirt comprising foldable extension members, according to an example, that protrude radially outward from the frame when the prosthetic heart valve is in a radially expanded state.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

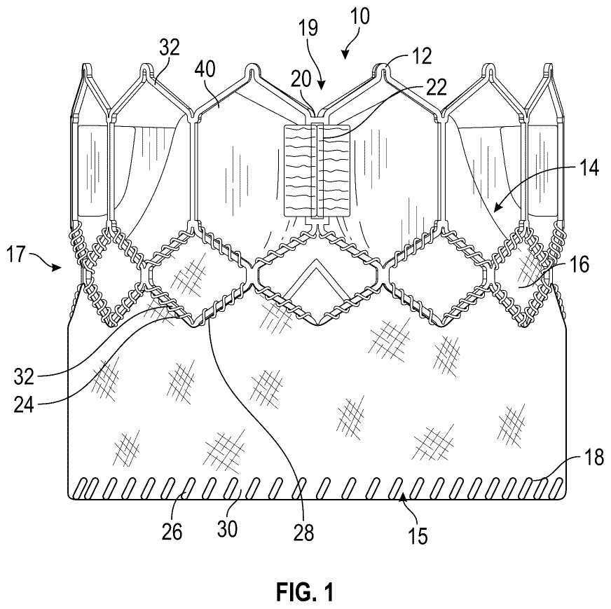

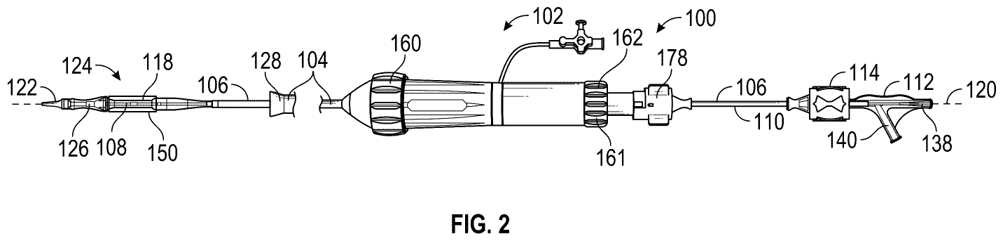

As introduced above, most prosthetic heart valves can include an outer skirt disposed around an outer surface of an annular frame of the prosthetic heart valve. The outer skirt can be configured to form a seal against native tissue upon implantation of the prosthetic heart valve, thereby reducing paravalvular leakage (PVL) past the prosthetic heart valve when expanded against the native anatomy. FIG. 1 illustrates an exemplary prosthetic device (prosthetic heart valve) comprising a frame, leaflets secured on an inside of the frame, and an outer skirt disposed around an outer surface of the frame. The prosthetic device can be advanced through a patient's vasculature, such as to a native heart valve, by a delivery apparatus, such as the exemplary delivery apparatus shown in FIG. 2. It may be desirable for the outer skirt of the prosthetic heart valve to be capable of extending radially outward (toward a surface of the surrounding anatomy) when the prosthetic heart valve is expanded at the native anatomy. However, billowing fabric or extension members of the outer skirt that are configured to extend radially outward can increase the overall crimp profile of the prosthetic heart valve, thereby causing a user advancing the prosthetic heart valve through the patient's vasculature to experience increased push forces.

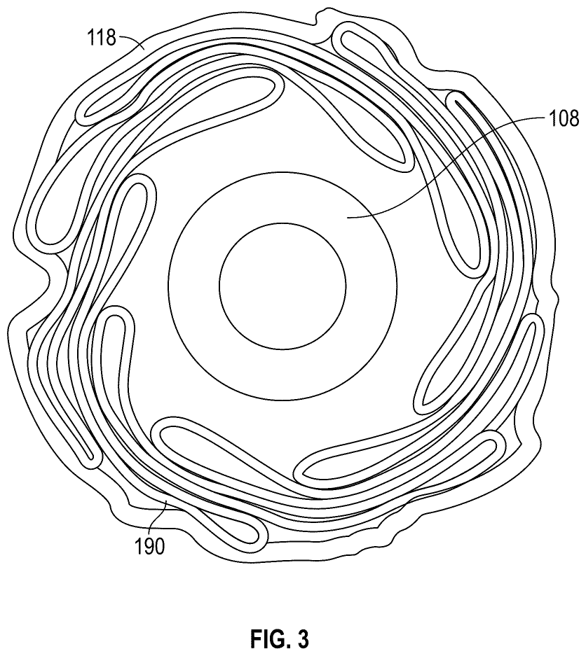

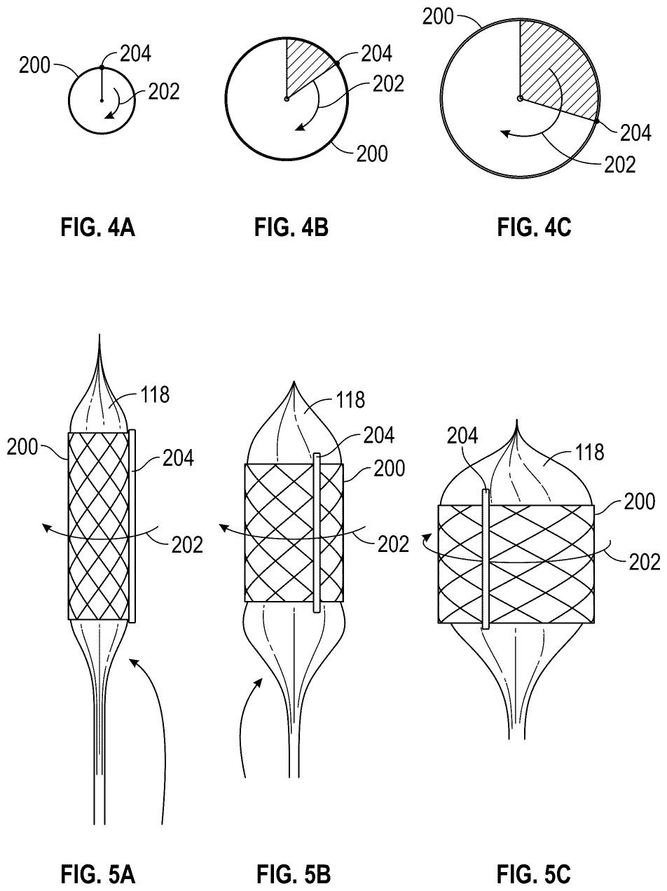

In some examples, the prosthetic heart valve can be mounted around an inflatable balloon of the delivery apparatus, where the inflatable balloon is deflated and folded around a shaft of the delivery apparatus during advancing the delivery apparatus through the patient's vasculature, as shown in FIG. 3. Upon reaching a target implantation site for the prosthetic heart valve, the balloon can be inflated, thereby radially expanding the prosthetic heart valve. As the balloon is inflated, the folds of the balloon can unravel, thereby causing the prosthetic heart valve to rotate in a first direction as it radially expands, as illustrated schematically in FIGS. 4A-4C and 5A-5C.

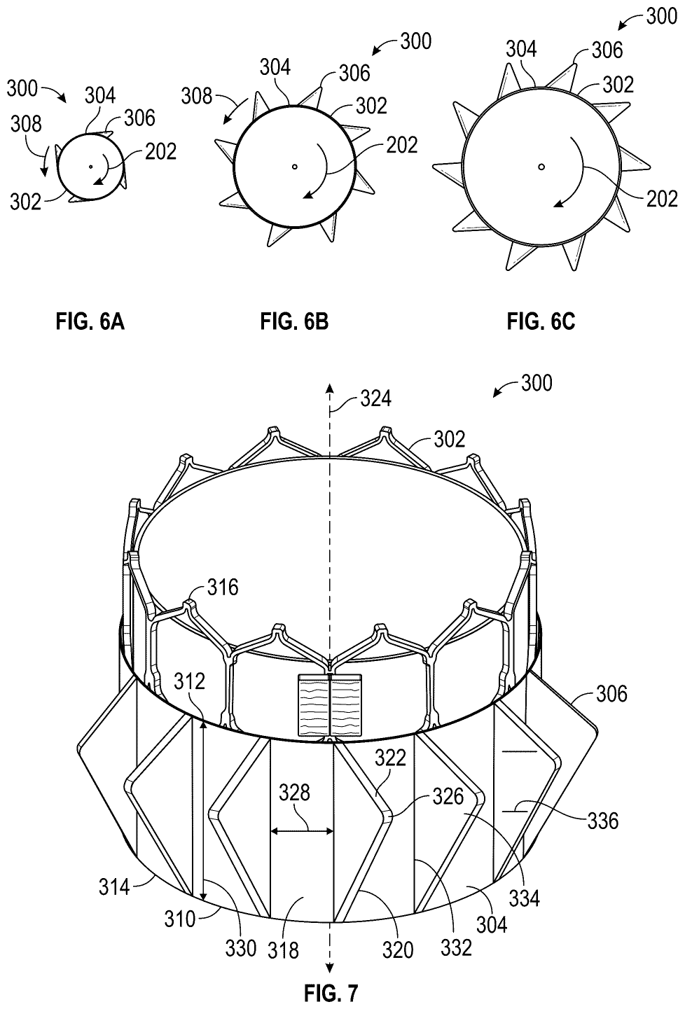

To take advantage of this valve rotation upon expansion, in some examples, the outer skirt of the prosthetic heart valve can include flexible extension members that are folded against the frame of the prosthetic heart valve when the prosthetic heart valve is in the radially compressed state and that are configured to unfold in a second direction that is opposite the first direction, as the prosthetic heart valve rotates in the first direction and radially expands, as illustrated in FIGS. 6A-6C. When the prosthetic heart valve is in the radially expanded state, the extension members can protrude radially outward and away from the frame of the prosthetic heart valve, as shown in FIGS. 7-9. As a result, the outer skirt can provide enhanced PVL sealing with the native anatomy, while also maintaining a relatively low crimp profile when the prosthetic heart valve is radially compressed around the delivery apparatus.

### Examples of the Disclosed Technology

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19.

The valvular structure 14 can comprise three leaflets 40, collectively forming a leaflet structure, which can be arranged to collapse in a tricuspid arrangement, although in other examples there can be greater or fewer number of leaflets (for example, one or more leaflets 40). The leaflets 40 can be secured to one another at their adjacent sides to form commissures 22 of the valvular (or leaflet) structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (for example, stainless steel, etc.) or self-expanding materials (e.g., Nitinol), as known in the art. When constructed of a plastically-expandable material, the frame 12 (and thus the prosthetic valve 10) can be crimped to a radially collapsed (or compressed) configuration on a delivery catheter and then expanded inside a patient by an inflatable balloon or equivalent expansion mechanism to a radially expanded configuration. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035comprises 35% nickel, 35% cobalt, 20% chromium, and 10% molybdenum, by weight.

The frame 12 can comprise a plurality of interconnected struts 32 that form open cells in the frame.

In some examples, as shown in FIG. 1 an upper edge portion 28 (also referred to as an outflow edge portion) of the outer skirt 18 can be secured to the frame 12 by stitches 24 and a lower edge portion 30 (also referred to as an inflow edge portion) of the outer skirt 18 can be secured to the frame 12 by stitches 26 extending along the inflow end portion 15 of the prosthetic valve 10. For example, the stitches 24 can wrap around struts 32 of the frame 12 forming a row of circumferentially extending struts 32 at the intermediate portion 17 of the prosthetic valve 10. Further, in some examples, the stitches 26 can wrap around struts 32 of the frame 12 forming a row of circumferentially extending struts 32 at the inflow end portion 15 of the prosthetic valve 10. In some instances, the upper edge portion 28 can be secured to struts 32 that are closer to the outflow end portion 19 of the frame 12 (such as the row of circumferentially extending struts 32 forming inflow ends of the row of cells disposed at the outflow end portion 19.

FIG. 2 shows a delivery apparatus 100, according to an example, that can be used to implant an expandable prosthetic heart valve (for example, the prosthetic heart valve 10 of FIG. 1 and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIGS. 2), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in PCT Application No. PCT/US2021/047056, which is incorporated by reference herein.

FIG. 3 is an exemplary, cross-sectional view of the balloon 118 of the delivery apparatus 100, wrapped and folded around the inner shaft 108, at the valve mounting portion 124 of the delivery apparatus 100. As shown in FIG. 3, the balloon 118 can include a plurality of overlapping pleats or folds 190 when in its deflated configuration and when a prosthetic valve is mounted on and radially compressed around the balloon 118. The balloon 118 can be folded in such a way that the pleats 190 result in a minimized folded balloon diameter (for example, in its deflated configuration) which can reduce a diameter of the radially compressed prosthetic valve when crimped thereon (referred to herein as the “crimp profile” of the prosthetic valve).

When the balloon 118 is inflated (for example, when the distal end portion of the delivery apparatus and the prosthetic valve have reached the target implantation site, such as the native valve), the balloon 118 unfurls (or unwraps) into its expanded state, thereby radially expanding the prosthetic valve to its radially expanded state. As the balloon 118 expands, and its folds or pleats 190 unwrap, the prosthetic valve radially expands and rotates. For example, as shown schematically by the cross-sectional and side views of FIGS. 4A-4C and 5A-5C, respectively, the unfolding of the pleats 190 of the balloon 118 causes a frame 200 of the prosthetic valve to rotate in a first direction 202 (first circumferential direction) during the balloon inflation. An imaginary axial line 204 is drawn on the frame 200 in FIGS. 4A-4C and 5A-5C to illustrate the rotation of the frame 200 during balloon inflation and radial expansion of the frame 200. As the balloon 118 expands, the frame 200 radially expands (expands in diameter) and the frame 200 and the axial line 204 rotate in the first direction 202. As such, the position of the radially expanded frame 200 of the prosthetic valve (FIGS. 4C and 5C) is rotated relative to the position of the radially compressed frame 200 (for example, when mounted on the delivery apparatus prior to inflating the balloon 118) (FIGS. 4A and 5A). It should be noted that the degree of rotation of the frame 200 may be exaggerated in FIGS. 4A-4C and/or FIGS. 5A-5C for the purpose of illustration.

As illustrated schematically in FIGS. 6A-6C, to take advantage of this rotation of the prosthetic valve upon radial expansion, an outer skirt 304 of a prosthetic heart valve 300 can include one or more flexible extension members 306 that are folded against the frame 302 of the prosthetic heart valve 300 when the prosthetic heart valve is in the radially compressed state or configuration (FIG. 6A). The extension members 306 can be configured to unfold in a second direction 308 that is opposite the first direction 202, as the prosthetic heart valve rotates in the first direction 202 and radially expands. In some instances, the flexible extension members of the outer skirt that are described herein can be configured to unfold from a folded state to an unfolded state upon radial expansion of the prosthetic heart valve, even if the prosthetic heart valve does not rotate during radial expansion (for example, if the prosthetic heart valve is mechanically expandable or self-expanding).

As shown in FIG. 6C, when the prosthetic heart valve 300 is in the radially expanded state or configuration, the extension members 306 (or unattached ends of the extension members 306) can protrude radially outward and away from the frame 302 of the prosthetic heart valve 300. As the prosthetic valve is expanded by the balloon, the extension members come into contact with the surrounding native tissue (for the example, the native annulus). As the extension members 306 come in contact with the surrounding tissue, the continued rotation of the valve in the first direction 202 causes the extension members 306 to rotate in the second direction 308, thereby resulting in an outwardly spread configuration of the extension members 306 (FIG. 6C) which enhances PVL sealing. For example, the extension members 306 can extend into and fills gaps between the native tissue at the implantation site and the prosthetic heart valve 300, thereby providing enhanced PVL sealing. At the same time, by being flexible and configured to fold against the frame 302 when the prosthetic heart valve 300 is in the radially compressed state (FIG. 6A), the extension members 306 can allow the prosthetic heart valve to maintain a reduced crimp profile (relative to skirts with non-foldable extension members or billowing material) that reduces push forces as the radially compressed prosthetic heart valve is navigated to an implantation site.

FIG. 7 shows the prosthetic heart valve 300 in the radially expanded state with the extension members 306 in their unfolded configuration. The outer skirt 304 is disposed around an outer surface of the frame 302. In some examples, an inflow edge 310 and an outflow edge 312 of the outer skirt 304 can be secured to the frame (for example, by sutures, similar to as shown in FIG. 1). Further in some examples, the inflow edge 310 can be disposed at and/or secured to an inflow end 314 of the frame 302 and the outflow edge 312 can be disposed at and/or secured to an intermediate portion of the frame 302 (which in some examples can be closer to an outflow end 316 of the frame 302 than the inflow end 314). The skirt 304 can be positioned at other locations along the height of the frame 302 and can be sized to cover other portions of the frame than what is shown in FIG. 7. For example, the skirt 304 can extend the entire height of the frame from the inflow end of the frame to the outflow end of the frame (for example, from the inflow portion 15 to the outflow portion 19 of the frame 12 in FIG. 1).

In some examples, the inflow edge 310 of the skirt can be axially offset from the inflow end 314 of the frame in the downstream direction (for example, a direction from the inflow end 314 to the outflow end 316).

In some examples, the frame 302 can be the frame 12 of FIG. 1. In alternate examples, the frame 302 can be a different type of frame for a radially expandable prosthetic heart valve.

The outer skirt 304 and other skirts or coverings described herein can comprise various synthetic materials, including fabrics (for example, polyethylene terephthalate fabric (PET fabric) or ultra high molecular weight polyethylene (UHMWPE) fabric)), polytetrafluoroethylene (PTFE), thermoplastic polyurethane (TPU), non-fabric materials or membranes (for example, made of TPU or other polymers), a hybrid material comprising one or more fabric layers and one or more non-fabric polymeric layers (for example, PET coated in TPU), or natural tissue (for example, pericardial tissue). In some examples, at least a main body portion 318 of the outer skirt 304 (the portion that lies relatively flat (and/or taut) against the outer surface of the frame 302, as shown in FIG. 7) can be formed of a relatively flat fabric which allows for a smaller crimp profile when the prosthetic heart valve 300 is in the radially compressed configuration.

The extension members 306 can comprise the same or a different material than the main body portion 318. In some examples, the outer skirt 304 can be formed from a single piece of fabric (for example, a PET fabric or another type of material) that is folded in the manner shown in FIG. 7 to form the extension members 306 and the main body portion 318.

In some examples, the extension members 306 can be formed from separate pieces of material that are joined to the main body portion 318, such as with sutures.

In some examples, the prosthetic valve 300 is crimped (for example, radially compressed utilizing a crimping tool) into the radially compressed configuration (for example, as shown in FIG. 6A) with the extension members 306 in the folded state against the frame 302. As the crimped prosthetic heart valve 300 is advanced to an implantation site on the delivery apparatus, the extension members 306 can hold their folded state since the frictional forces of the prosthetic heart valve sliding against an introducer sheath (disposed in a patient's vasculature, through which the delivery apparatus is advanced) acts along the axial direction which is generally perpendicular to the direction of the folds of the extension members 306. As described above, the rotation of the prosthetic heart valve 300 in the circumferential direction as it is radially expanded causes the extension members 306 to come into contact with the surrounding tissue and unfold from the folded state to the unfolded state.

In some examples, the extension members 306 can comprise one or more metal wires or struts 336 embedded within or attached to the material (for example, fabric) of the extension members 306, as shown in FIG. 7. In some examples, the struts 336 can comprise a shape memory material, such as Nitinol, or a plastically deformable metal, such as stainless steel or a cobalt chromium alloy. The struts 336 can be shaped or bent in the circumferential direction, against or close to the main body portion 318 and the frame 302 when the extension members are folded. The rigidity of the struts 336 can assist in holding the fabric of the extension members 306 in the folded state. In instances where the struts 336 comprise Nitinol, the struts 336 can be shape set into the folded state and then deformed when the frame 302 is radially expanded by the contact of the extension members 306 with the native tissue (valve annuls) as the prosthetic heart valve 300 rotates (as described above with reference to FIGS. 6A-6C).

In some instances, the extension members 306 can be self-expandable from the folded state to the unfolded state. As such, the extension members 306 can be folded into the folded state and then held in the folded state, around the radially compressed prosthetic heart valve 300 that is mounted on the delivery apparatus, by a removable element or restraint that surrounds at least a portion of the outer skirt 304 (and the prosthetic heart valve 300). For example, the removable element can be a delivery capsule, belt, or lasso-type device that extends around at least a portion of the outer skirt 304 and can be removed prior to radial expansion of the prosthetic heart valve 300. Upon removal of the removable element and during radial expansion and rotation of the prosthetic heart valve 300, the extension members 306 can then unfold and expand to their unfolded state. The self-expansion of the extension members 306 can be achieved by forming the extension members from a fabric and shape setting the extension members in the unfolded state. In lieu of or in addition to shape setting the fabric material, the wires or struts 336 can be formed from a shape-memory material (for example, Nitinol) and the wires or struts 336 can be shape set in the unfolded state in which the wires or struts extend radially outwardly from the frame 302 of the prosthetic valve.

In some examples, the outer skirt 304 can comprise a plurality of extension members 306 that are spaced circumferentially apart from one another around the outer skirt 304. A number of the extension members 306 and a spacing 328 between adjacent extension members 306 can be specified based on the target implantation site (and, for example, a desired amount of PVL sealing). In some examples, the spacing 328 can be specified such that the extension members 306 do not overlap one another (or minimally overlap one another) in the folded state (FIG. 6A), thereby reducing the crimp profile of the prosthetic heart valve 300. In some examples, the spacing 328 is larger than a thickness of the extension members 306 (for example, the thickness can be measured in the circumferential direction, across the extension members 306, when the extension members 306 extend radially outward from the frame, as shown in FIG. 7). In some example, the spacing 328 between adjacent extension members 306 can be varied between different locations of the outer skirt 304. The spacing 328 associated with a first set of extension members 306 located at a first location of the outer skirt 304 can be greater or less than the spacing 328 associated with a second set of extension members 306 located at a second location of the outer skirt 304. The first location and the second location can be different circumferential locations or axial locations (for example, inflow edge, outflow edge, intermediate portion between the inflow edge and the outflow edge, etc.) of the prosthetic heart valve 300.

The extension members 306 can have an angled shape where a first or lower edge 320 and/or a second or upper edge 322 of each extension member 306 is angled relative to a central longitudinal axis 324 of the prosthetic heart valve 300. For example, as shown in FIG. 7, the lower edge 320 angles radially outward from the main body portion 318 at the inflow edge 310 of the outer skirt 304 to a pointed tip 326 (or apex) of the extension member 306. Similarly, the upper edge 322 can angle radially outward from the main body portion 318 at the outflow edge 312 of the outer skirt 304 to the pointed tip 326. In this way, the extension members 306 can have a triangular shape.

In alternate examples, the pointed tip 326 can be closer to the inflow edge 310 or the outflow edge 312 of the outer skirt 304 than shown in FIG. 7.

Each extension member 306 can have an attached end 332 (base) and an unattached end 334, where the attached end 332 is attached to or continuous with the main body portion 318 and the unattached end 334 is configured to fold against (in the folded state) and extend away from (in the unfolded state) the main body portion 318.

As shown in FIG. 7, each extension member 306 can span an entire height 330 of the outer skirt 304. In alternate examples, one or more of the extension members 306 can span only a portion of the height 330 of the outer skirt 304. For example, one or more extension members 306 can span an intermediate portion of the outer skirt 304 without extending to the inflow edge 310 and/or the outflow edge 312.

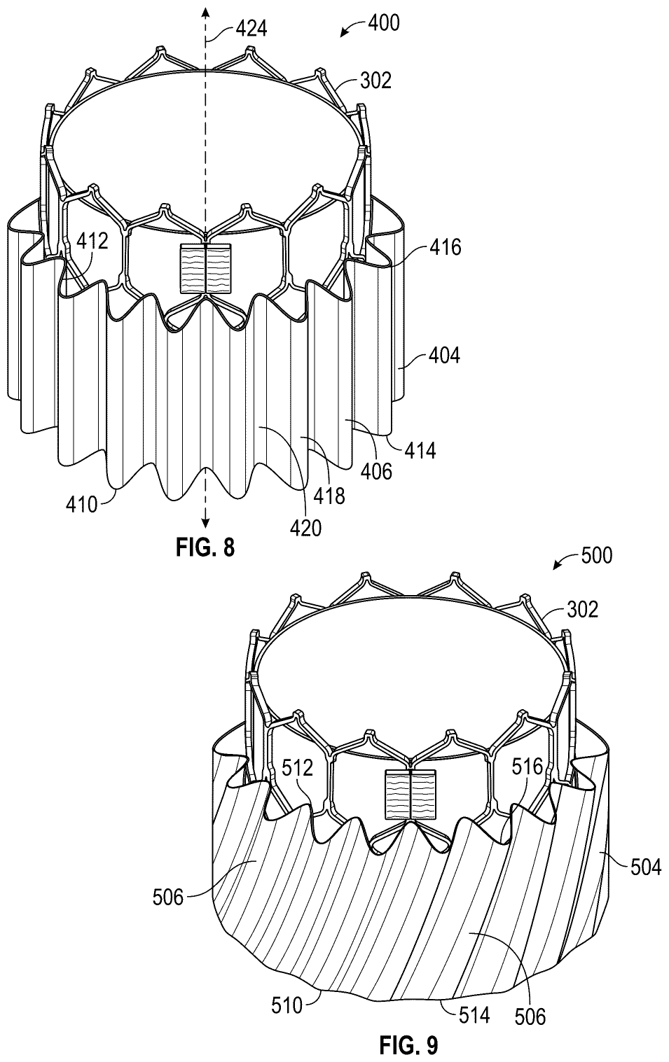

FIGS. 8 and 9 show additional examples of extension members for an outer skirt of a prosthetic heart valve. The extension members shown in FIGS. 8 and 9 can be configured to operate similarly to extension members 306, as described above, but have a different structure. Additionally, the extension members shown in FIGS. 8 and 9 can be used with outer skirts for the frame 302 (as shown in FIGS. 8 and 9) or the frame 12 of FIG. 1.

For example, FIG. 8 shows a prosthetic heart valve 400 in the radially expanded state with an outer skirt 404 disposed around an outer surface of the frame 302. The outer skirt 404 comprises a plurality of extension members 406 extending between an inflow edge 410 and an outflow edge 412 of the outer skirt 404. The extension members 406 can be flexible and configured to fold into a folded state (against the frame 302) when the prosthetic heart valve 400 is radially compressed (similar to as shown in FIG. 6A) and unfold into an unfolded state where they protrude radially outward and away from the frame 302 when the prosthetic heart valve 400 is radially expanded (FIG. 8).

As shown in FIG. 8, each of the extension members 406 can extend longitudinally (relative to a central longitudinal axis 424 of the prosthetic heart valve 400) between the inflow edge 410 and the outflow edge 412. In some examples, each extension member 406 can have a substantially uniform profile between its inflow end 414 and outflow end 416. In alternate examples, a cross-sectional profile of each extension member 406, between its inflow end 414 and outflow end 416, may not be uniform (for example, the extension member 406 can be wider at its inflow end 414 than its outflow end 416 or vice versa, or the extension member 406 can be wider at its middle portion than at its inflow end 414 and outflow end 416).

The extension members 406 can be spaced circumferentially apart from one another around the outer skirt 404. In some examples, the spacing between adjacent extension members 406 can be uniform. In alternate examples, the spacing between adjacent extension members 406 can vary around the outer skirt 404, as described herein with respect to the extension members 306 of the outer skirt 304.

In some examples, the extension members 406 can be continuous with one another around the outer skirt 404, forming an undulating pattern of peaks 418 (most radially outwardly protruding points) that form the extension members and valleys 420 (disposed against the frame 302) that extend between adjacent extension members. In this way, the outer skirt 404 can be formed as one piece and the extension members 406 are continuous with (or form) a main body portion of the outer skirt 404 that is disposed against (flush with) the outer surface of the frame 302. The peaks 418 and valleys 420 can be formed by connecting the skirt 404 to the frame 302 at locations along the longitudinal center lines of the valleys 420 (that is, along lines parallel to the central axis 424 extending along the valleys), such as with one or more sutures forming stitches that extend through the outer skirt and around selected struts of the frame.

In some examples, the extension members 406 can be separated from one another and each protrude radially outward from a main body portion of the outer skirt 404 (for example, similar to as shown in FIG. 7, but having the shape shown in FIG. 8).

FIG. 9 shows a prosthetic heart valve 500 in the radially expanded state with an outer skirt 504 disposed around an outer surface of the frame 302. The outer skirt 504 comprises a plurality of extension members 506 extending between an inflow edge 510 and an outflow edge 512 of the outer skirt 504. The extension members 506 can be flexible and configured to fold into a folded state (against the frame 302) when the prosthetic heart valve 500 is radially compressed (similar to as shown in FIG. 6A) and unfold or unfurl into an unfolded state where they protrude radially outward and away from the frame 302 when the prosthetic heart valve 500 is radially expanded (FIG. 9).

Each of the extension members 506 can be curved between its inflow end 514 and outflow end 516 (such that the inflow end 514 and outflow end 516 of the extension member are circumferentially offset from each other). Thus, each extension member 506 can have a partial helix shape. Similar to the outer skirt 404 of FIG. 8, the outer skirt 504 can include a plurality of peaks and valleys, with the peaks forming the extension members 506. The peaks and valleys can be formed by connecting the outer skirt to the frame at locations along the valleys (along a helical or spiral path), such as with one or more sutures forming stitches that extend through the outer skirt and around selected struts of the frame.

In some examples, a cross-sectional profile of one of more of the extension members 506, between their inflow end 514 and outflow end 516, can vary. For example, one or more extension members 506 can be wider at the inflow end 514 than the outflow end 516 or vice versa.

In some examples, one or more of the extension members 506 can be wider at its middle portion than at the inflow end 514 and the outflow end 516.

In some examples, a spacing between adjacent extension member 506 can vary around the outer skirt 504, as described herein with respect to the extension members 306 of the outer skirt 304. Additionally or alternatively, the spacing between adjacent extension members 506 can vary between the inflow end 514 and the outflow end 516 of the adjacent extension members 506. For example, in some instances, the spacing between adjacent extension members 506 can be larger at one or both of the inflow end 514 and the outflow end 516 than at the middle portion of the extension members 506.

In some instances, the spacing between adjacent extension members 506 can be smaller at the outflow ends 516 of the extension members 506 than at the inflow ends 514.

The configuration of FIG. 9 advantageously combines both the counter-rotational unfolding effect described above with reference to FIGS. 6A-6C, and the effect of the frame 302 foreshortening as it radially expands, thereby allowing the extension members 506 to stretch to an even lower profile (in the radial direction) when the prosthetic heart valve 500 is radially compressed. This can further reduce the crimp profile of the radially compressed prosthetic heart valve 500.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta. around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively. in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame that is radially expandable and compressible between a radially compressed configuration and a radially expanded configuration; and an outer skirt disposed around an outer surface of the frame, wherein the outer skirt comprises one or more extension members that are movable between a first state where the one or more extension members are folded against the frame and a second state where the one or more extension members extend radially outward and away from the frame, and wherein the one or more extension members are in the first state when the frame is in the radially compressed configuration and in the second state when the frame is in the radially expanded configuration.

Example 2. The prosthetic heart valve of any example herein, particularly example 1, wherein the one or more extension members comprise a flexible material and are configured to unfold from the first state to the second state as the frame radially expands from the radially compressed configuration to the radially expanded configuration.

Example 3. The prosthetic heart valve of any example herein, particularly example 2, wherein, in the first state, the one or more extension members are folded against the frame in a first direction, wherein the one or more extension members are configured to unfold in a second direction that is opposite the first direction upon moving from the first state to the second state, and wherein the prosthetic heart valve is configured to rotate in the first direction as it transitions between the radially compressed configuration and the radially expanded configuration by inflation of a balloon.

Example 4. The prosthetic heart valve of any example herein, particularly any one of examples 1-3, wherein the one or more extension members comprise a fabric material and one or more metal struts embedded within or attached to the fabric material.

Example 5. The prosthetic heart valve of any example herein, particularly any one of examples 1-4, wherein the one or more extension members includes a plurality of extension members spaced circumferentially apart around the outer skirt, and wherein all extension members of the plurality of extension members are folded in a same direction against the frame in the first state.

Example 6. The prosthetic heart valve of any example herein, particularly example 5, wherein the extension members of the plurality of extension members are continuous with one another around the outer skirt, and wherein, in the second state, each extension member of the plurality of extension members forms a peak disposed radially outward and away from the frame, and valleys in the outer skirt are formed between adjacent peaks, the valleys disposed against the frame.

Example 7. The prosthetic heart valve of any example herein, particularly example 6, wherein each extension member of the plurality of extension members extends longitudinally, in parallel to a central longitudinal axis of the prosthetic heart valve, from an inflow edge to an outflow edge of the outer skirt.

Example 8. The prosthetic heart valve of any example herein, particularly example 6, wherein each extension member of the plurality of extension members extends, at a non-zero angle relative to a central longitudinal axis of the prosthetic heart valve, from an inflow edge to an outflow edge of the outer skirt such that an inflow end and outflow end of each extension member are circumferentially offset from one another.

Example 9. The prosthetic heart valve of any example herein, particularly any one of examples 1-5, wherein each extension member of the one or more extension members has at least one edge that angles radially outward from a main body portion of the outer skirt that is disposed against the frame, and wherein the angle is non-zero relative to a central longitudinal axis of the prosthetic heart valve.

Example 10. The prosthetic heart valve of any example herein, particularly any one of examples 1-5, wherein each extension member of the one or more extension members comprises: a first edge that angles radially outward from the main body portion at an inflow edge of the outer skirt to an apex of the extension member that is disposed radially outward and away from the main body portion; and a second edge that angles radially outward from the main body portion at an outflow edge of the outer skirt to the apex.

Example 11. The prosthetic heart valve of any example herein, particularly any one of examples 1-10, wherein the outer skirt comprises an inflow edge secured to an inflow end of the frame and an outflow edge secured to an intermediate portion of the frame that is disposed between the inflow end and an outflow end of the frame.

Example 12. The prosthetic heart valve of any example herein, particularly example 11, wherein each extension member of the one or more extension members extends between the inflow edge and the outflow edge of the outer skirt.

Example 13. The prosthetic heart valve of any example herein, particularly any one of examples 1-12, further comprising a plurality of leaflets arranged within an interior of the frame.

Example 14. A prosthetic heart valve comprising: a radially expandable and compressible annular frame configured to move between a radially compressed state and a radially expanded state; and an outer skirt disposed around an outer surface of the frame, the outer skirt comprising: a first portion disposed against the outer surface of the frame; and a second portion comprising a plurality of extension members spaced circumferentially apart from each other around the outer skirt, wherein the plurality of extension members is configured to be folded against the first portion when the frame is in the radially compressed state and unfold to extend radially outward from the frame when the frame is radially expanded from the radially compressed state to the radially expanded state.

Example 15. The prosthetic heart valve of any example herein, particularly example 14, wherein an outflow edge and an inflow edge of the first portion of the outer skirt are secured to the frame.

Example 16. The prosthetic heart valve of any example herein, particularly example 15, wherein each extension member of the plurality of extension members extends between the outflow edge and the inflow edge of the first portion.

Example 17. The prosthetic heart valve of any example herein, particularly example 15 or example 16, wherein the inflow edge is secured to an inflow end of the frame and the outflow edge is secured to an intermediate portion of the frame that is disposed between the inflow end and an outflow end of the frame.

Example 18. The prosthetic heart valve of any example herein, particularly any one of examples 14-17, wherein the first portion is disposed flush against the frame, and wherein the plurality of extension members is configured to be folded against the first portion in a single direction around the outer skirt.

Example 19. The prosthetic heart valve of any example herein, particularly any one of examples 14-18, wherein a spacing between adjacent extension members of the plurality of extension members is greater than a thickness of the plurality of extension members.

Example 20. The prosthetic heart valve of any example herein, particularly any one of examples 14-19, wherein each extension member of the plurality of extension members is triangular with a first angled edge extending radially outward from an inflow edge of the first portion and a second angled edge extending radially outward from an outflow edge of the first portion, the first angled edge and second angled edge meeting at an apex disposed radially outward and away from the first portion.

Example 21. The prosthetic heart valve of any example herein, particularly any one of examples 14-19, wherein the extension members of the plurality of extension members are continuous with one another, and wherein the first portion is formed at valleys between adjacent extension members.

Example 22. The prosthetic heart valve of any example herein, particularly example 21, wherein each extension member of the plurality of extension members extends between an inflow edge and an outflow edge of the outer skirt, in a longitudinal direction that is parallel to a central longitudinal axis of the frame.

Example 23. The prosthetic heart valve of any example herein, particularly example 21, wherein each extension member of the plurality of extension members extends between an inflow edge and an outflow edge of the outer skirt, at a non-zero angle relative to a central longitudinal axis of the frame.

Example 24. The prosthetic heart valve of any example herein, particularly any one of examples 14-23, wherein the first portion and the second portion comprise a same material.

Example 25. The prosthetic heart valve of any example herein, particularly any one of examples 14-24, wherein the outer skirt comprises a fabric, wherein each extension member of the plurality of extension members comprises one or more metal struts embedded within or attached to the fabric of the extension member, and wherein the one or more metal struts are configured to hold the one or more extension members against the first portion when the frame is in the radially compressed state and deform to extend radially outward from the frame when the frame is radially expanded from the radially compressed state to the radially expanded state.

Example 26. The prosthetic heart valve of any example herein, particularly any one of examples 14-25, further comprising a plurality of leaflets arranged within an interior of the frame.

Example 27. A method comprising: advancing a prosthetic heart valve that is radially compressed around an inflatable balloon of a distal end portion of a delivery apparatus to an implantation site using the delivery apparatus, wherein the prosthetic heart valve comprises an annular frame and an outer skirt disposed around an outer surface of the frame, wherein the outer skirt comprises extension members that are folded against the frame; and inflating the balloon of the delivery apparatus at the implantation site to radially expand and implant the prosthetic heart valve, and unfolding the extension members to protrude radially outward and away from the frame as the prosthetic heart valve radially expands.

Example 28. The method of any example herein, particularly example 27, wherein the inflating the balloon includes rotating the prosthetic heart valve in a first direction as the balloon inflates and the prosthetic heart valve radially expands, and wherein the extension members unfold in a second direction that is opposite the first direction.

Example 29. The method of any example herein, example 28, wherein when the prosthetic heart valve is radially compressed around the balloon, the extension members are folded against the frame in the first direction.

Example 30. The method of any example herein, particularly any one of examples 27-29, wherein the frame and the outer skirt shorten in an axial direction as the prosthetic heart valve radially expands.

Example 31. The method of any example herein, particularly any one of examples 27-30, wherein the unfolded extension members extend into and fill gaps between native tissue at the implantation site and the prosthetic heart valve.

Example 32. The method of any example herein, particularly any one of examples 27-31, wherein the extension members are spaced circumferentially apart around the outer skirt, and wherein the extension members comprise a flexible material.

Example 33. The method of any example herein, particularly any one of examples 27-32, wherein the extension members are continuous with one another around the outer skirt, and wherein, when the prosthetic heart valve is radially expanded, each extension member forms a peak disposed radially outward and away from the frame, and valleys in the outer skirt are formed between adjacent peaks, the valleys disposed against the frame.

Example 34. The method of any example herein, particularly any one of examples 27-33, wherein each extension member extends longitudinally, in parallel to a central longitudinal axis of the prosthetic heart valve, from an inflow edge to an outflow edge of the outer skirt.

Example 35. The method of any example herein, particularly any one of examples 27-33, wherein each extension member extends, at a non-zero angle relative to a central longitudinal axis of the prosthetic heart valve, from an inflow edge to an outflow edge of the outer skirt such that an inflow end and outflow end of each extension member are circumferentially offset from one another.

Example 36. The method of any example herein, particularly any one of examples 37-35, wherein each extension member comprises: a first edge that angles radially outward from a main body portion of the outer skirt that is disposed against the frame, at an inflow edge of the outer skirt, to an apex of the extension member that is disposed radially outward and away from the main body portion; and a second edge that angles radially outward from the main body portion at an outflow edge of the outer skirt to the apex.

Example 37. The method of any example herein, particularly any one of 27-36,wherein the outer skirt comprises an inflow edge secured to an inflow end of the frame and an outflow edge secured to an intermediate portion of the frame that is disposed between the inflow end and an outflow end of the frame.

Example 38. The method of any example herein, particularly example 37, wherein each extension member extends between the inflow edge and the outflow edge of the outer skirt.

Example 39. An assembly comprising: an inflatable balloon disposed at a distal end portion of a delivery apparatus; and a prosthetic heart valve mounted around the balloon in a radially compressed configuration, wherein the prosthetic heart valve comprises: an annular frame; and an outer skirt disposed around an outer surface of the frame, the outer skirt comprising a plurality of extension members spaced circumferentially apart from each other around the outer skirt, wherein the plurality of extension members are folded against the frame when the frame is in the radially compressed configuration and are configured to unfold to extend radially outward from the frame when the frame is radially expanded from the radially compressed configuration to a radially expanded configuration by inflation of the balloon.

Example 40. The assembly of any example herein, particularly example 39, wherein the inflatable balloon comprises a plurality of overlapping folds in a deflated state, the prosthetic heart valve mounted around the plurality of overlapping folds of the balloon in the radially compressed configuration, and wherein the inflatable balloon is configured to unfold as it inflates which causes the prosthetic heart valve to rotate in a first direction as it radially expands into the radially expanded configuration.

Example 41. The assembly of any example herein, particularly example 40, wherein the plurality of extension members is folded against the frame in the first direction, and wherein the plurality of extension members is configured to unfold in a second direction that is opposite the first direction, as the prosthetic heart valve rotates in the first direction and is radially expanded to the radially expanded configuration.

Example 42. The assembly of any example herein, particularly any one of examples 39-41, wherein the extension members are continuous with one another around the outer skirt, and wherein each extension member forms a radially outwardly protruding peak that extends longitudinally, in parallel to a central longitudinal axis of the prosthetic heart valve, from an inflow edge to an outflow edge of the outer skirt.

Example 43. The assembly of any example herein, particularly any one of examples 39-41, wherein the extension members are continuous with one another around the outer skirt, and wherein each extension member forms a radially outwardly protruding peak that extends, at a non-zero angle relative to a central longitudinal axis of the prosthetic heart valve, from an inflow edge to an outflow edge of the outer skirt such that an inflow end and outflow end of each extension member are circumferentially offset from one another.

Example 44. The assembly of any example herein, particularly any one of examples 39-41, wherein each extension member of the plurality of extension members has at least one edge that angles radially outward from a main body portion of the outer skirt that is disposed against the frame, wherein the angling is at a non-zero angle relative to a central longitudinal axis of the prosthetic heart valve.

Example 45. The assembly of any example herein, particularly example 44, wherein each extension member comprises: a first edge that angles radially outward from the main body portion at an inflow edge of the outer skirt to an apex of the extension member that is disposed radially outward and away from the main body portion; and a second edge that angles radially outward from the main body portion at an outflow edge of the outer skirt to the apex.

Example 46. The assembly of any example herein, particularly any one of examples 39-45, wherein the outer skirt comprises an inflow edge secured to an inflow end of the frame and an outflow edge secured to an intermediate portion of the frame that is disposed between the inflow end and an outflow end of the frame.

Example 47. The assembly of any example herein, particularly example 46, wherein each extension member of the plurality of extension members extends between the inflow edge and the outflow edge of the outer skirt.

Example 48. The assembly of any example herein, particularly any one of examples 39-47, wherein the prosthetic heart valve further comprises a plurality of leaflets arranged within an interior of the frame.

Example 49. A method comprising: radially expanding and rotating a prosthetic heart valve in a first direction as it radially expands by inflating a balloon of a delivery apparatus, wherein the prosthetic heart valve is mounted around the balloon in a radially compressed configuration prior to inflating the balloon, and wherein the prosthetic heart valve comprises an annular frame and an outer skirt disposed around an outer surface of the frame, the outer skirt comprising extension members that are folded against the frame; and unfolding the extension members in a second direction that is opposite the first direction in response to the rotating such that the extension members protrude radially outward from the frame when the prosthetic heart valve in a radially expanded configuration.

Example 50. The method of any example herein, example 49, wherein each extension member of the extension members comprises an attached end attached to or continuous with a main body portion of the outer skirt that is disposed against the frame, and an unattached end that transitions between a first state where the unattached end is disposed against the main body portion when the extension member is folded against the frame and a second state where the unattached end protrudes radially outward and away from the main body portion and the frame.

Example 51. The method of any example herein, particularly example 49 or example 50, wherein all extension members of the extension members are folded against the frame in the first direction when the prosthetic heart valve is in the radially compressed configuration.

Example 52. The method of any example herein, particularly any one of examples 49-51, wherein the frame and the outer skirt shorten in an axial direction during the radially expanding the prosthetic heart valve.

Example 53. The method of any example herein, particularly any one of examples 49-52, wherein the radially expanding includes radially expanding and implanting the prosthetic heart valve at an implantation site, and wherein the unfolded extension members extend into and fill gaps between native tissue at the implantation site and the radially expanded prosthetic heart valve.

Example 54. The method of any example herein, particularly any one of examples 49-53, wherein the extension members comprise a plurality of extension members that are spaced circumferentially apart around the outer skirt, and wherein the extension members are configured to flex between a folded state and unfolded state and remain in the unfolded state following radially expanding the prosthetic heart valve.

Example 55. The method of any example herein, particularly any one of examples 49-54, wherein the extension members are continuous with one another around the outer skirt, and wherein, when the prosthetic heart valve is radially expanded, each extension member forms a peak disposed radially outward and away from the frame, and valleys in the outer skirt are formed between adjacent peaks, the valleys disposed against the frame.

Example 56. The method of any example herein, particularly any one of examples 49-55, wherein each extension member extends longitudinally, in parallel to a central longitudinal axis of the prosthetic heart valve, from an inflow edge to an outflow edge of the outer skirt.

Example 57. The method of any example herein, particularly any one of examples 49-55, wherein each extension member extends, at a non-zero angle relative to a central longitudinal axis of the prosthetic heart valve, from an inflow edge to an outflow edge of the outer skirt such that an inflow end and outflow end of each extension member are circumferentially offset from one another.

Example 58. The method of any example herein, particularly any one of examples 49-54, wherein each extension member comprises: a first edge that angles radially outward from a main body portion of the outer skirt that is disposed against the frame, at an inflow edge of the outer skirt, to an apex of the extension member that is disposed radially outward and away from the main body portion; and a second edge that angles radially outward from the main body portion at an outflow edge of the outer skirt to the apex.

Example 59. The method of any example herein, particularly any one of examples 49-58, wherein the outer skirt comprises an inflow edge secured to an inflow end of the frame and an outflow edge secured to an intermediate portion of the frame that is disposed between the inflow end and an outflow end of the frame, and wherein each extension member extends between the inflow edge and the outflow edge of the outer skirt.

Example 60. The method of any example herein, particularly any one of examples 49-59, wherein the radially expanding and rotating the prosthetic heart valve occurs during implantation at a native valve annulus, wherein each of the extension members comprise a fabric and one or more metal struts attached to or embedded within the fabric, and wherein unfolding the extension members comprises deforming the one or more metal struts of each extension member as the extension members contact the native valve annulus as the prosthetic heart valve rotates.

Example 61. A prosthetic heart valve of any one of examples 1-60, wherein the prosthetic heart valve is sterilized.

Example 62. A method comprising sterilizing the prosthetic heart valve, apparatus, and/or assembly of any example.

The features described herein with regard to any example can be combined with other features described in any one or more of the other examples, unless otherwise stated. For example, any one or more of the features of one skirt for a prosthetic heart valve can be combined with any one or more features of another skirt for a prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame that is radially expandable and compressible between a radially compressed configuration and a radially expanded configuration; and
an outer skirt disposed around an outer surface of the frame, wherein the outer skirt comprises one or more extension members that are movable between a first state where the one or more extension members are folded against the frame and a second state where the one or more extension members extend radially outward and away from the frame, and wherein the one or more extension members are in the first state when the frame is in the radially compressed configuration and in the second state when the frame is in the radially expanded configuration.

2. The prosthetic heart valve of claim 1, wherein the one or more extension members comprise a flexible material and are configured to unfold from the first state to the second state as the frame radially expands from the radially compressed configuration to the radially expanded configuration.

3. The prosthetic heart valve of claim 2, wherein, in the first state, the one or more extension members are folded against the frame in a first direction, wherein the one or more extension members are configured to unfold in a second direction that is opposite the first direction upon moving from the first state to the second state, and wherein the prosthetic heart valve is configured to rotate in the first direction as it transitions between the radially compressed configuration and the radially expanded configuration by inflation of a balloon.

4. The prosthetic heart valve of claim 1, wherein the one or more extension members comprise a fabric material and one or more metal struts embedded within or attached to the fabric material.

5. The prosthetic heart valve of claim 1, wherein the one or more extension members includes a plurality of extension members spaced circumferentially apart around the outer skirt, and wherein all extension members of the plurality of extension members are folded in a same direction against the frame in the first state.

6. The prosthetic heart valve of claim 5, wherein the extension members of the plurality of extension members are continuous with one another around the outer skirt, and wherein, in the second state, each extension member of the plurality of extension members forms a peak disposed radially outward and away from the frame, and valleys in the outer skirt are formed between adjacent peaks, the valleys disposed against the frame.

7. The prosthetic heart valve of claim 1, wherein each extension member of the one or more extension members has at least one edge that angles radially outward from a main body portion of the outer skirt that is disposed against the frame, and wherein the angle is non-zero relative to a central longitudinal axis of the prosthetic heart valve.

8. The prosthetic heart valve of claim 1, wherein the outer skirt comprises an inflow edge secured to an inflow end of the frame and an outflow edge secured to an intermediate portion of the frame that is disposed between the inflow end and an outflow end of the frame.

9. The prosthetic heart valve of claim 8, wherein each extension member of the one or more extension members extends between the inflow edge and the outflow edge of the outer skirt.

10. An assembly comprising:
an inflatable balloon disposed at a distal end portion of a delivery apparatus; and
a prosthetic heart valve mounted around the balloon in a radially compressed configuration, wherein the prosthetic heart valve comprises:
an annular frame; and
an outer skirt disposed around an outer surface of the frame, the outer skirt comprising a plurality of extension members spaced circumferentially apart from each other around the outer skirt, wherein the plurality of extension members are folded against the frame when the frame is in the radially compressed configuration and are configured to unfold to extend radially outward from the frame when the frame is radially expanded from the radially compressed configuration to a radially expanded configuration by inflation of the balloon.

11. The assembly of claim 10, wherein the inflatable balloon comprises a plurality of overlapping folds in a deflated state, the prosthetic heart valve mounted around the plurality of overlapping folds of the balloon in the radially compressed configuration, and wherein the inflatable balloon is configured to unfold as it inflates which causes the prosthetic heart valve to rotate in a first direction as it radially expands into the radially expanded configuration.

12. The assembly of claim 11, wherein the plurality of extension members is folded against the frame in the first direction, and wherein the plurality of extension members is configured to unfold in a second direction that is opposite the first direction, as the prosthetic heart valve rotates in the first direction and is radially expanded to the radially expanded configuration.

13. The assembly of claim 10, wherein each extension member of the plurality of extension members has at least one edge that angles radially outward from a main body portion of the outer skirt that is disposed against the frame, wherein the angling is at a non-zero angle relative to a central longitudinal axis of the prosthetic heart valve.

14. The assembly of claim 10, wherein the outer skirt comprises an inflow edge secured to an inflow end of the frame and an outflow edge secured to an intermediate portion of the frame that is disposed between the inflow end and an outflow end of the frame.

15. A method comprising:
radially expanding and rotating a prosthetic heart valve in a first direction as it radially expands by inflating a balloon of a delivery apparatus, wherein the prosthetic heart valve is mounted around the balloon in a radially compressed configuration prior to inflating the balloon, and wherein the prosthetic heart valve comprises an annular frame and an outer skirt disposed around an outer surface of the frame, the outer skirt comprising extension members that are folded against the frame; and
unfolding the extension members in a second direction that is opposite the first direction in response to the rotating such that the extension members protrude radially outward from the frame when the prosthetic heart valve in a radially expanded configuration.

16. The method of claim 15, wherein each extension member of the extension members comprises an attached end attached to or continuous with a main body portion of the outer skirt that is disposed against the frame, and an unattached end that transitions between a first state where the unattached end is disposed against the main body portion when the extension member is folded against the frame and a second state where the unattached end protrudes radially outward and away from the main body portion and the frame.

17. The method of claim 15, wherein all extension members of the extension members are folded against the frame in the first direction when the prosthetic heart valve is in the radially compressed configuration.

18. The method of claim 15, wherein the radially expanding includes radially expanding and implanting the prosthetic heart valve at an implantation site, and wherein the unfolded extension members extend into and fill gaps between native tissue at the implantation site and the radially expanded prosthetic heart valve.

19. The method of claim 15, wherein the extension members comprise a plurality of extension members that are spaced circumferentially apart around the outer skirt, and wherein the extension members are configured to flex between a folded state and unfolded state and remain in the unfolded state following radially expanding the prosthetic heart valve.

20. The method of claim 15, wherein the outer skirt comprises an inflow edge secured to an inflow end of the frame and an outflow edge secured to an intermediate portion of the frame that is disposed between the inflow end and an outflow end of the frame, and wherein each extension member extends between the inflow edge and the outflow edge of the outer skirt.
